(19) United States
(12) Patent Application Publication
(10) Pub. No.: US 2009/0112064 A1
(43) Pub. Date: Apr. 30, 2009

(54) Endoscopic Lubricating and Gripping Device

(76) Inventors: John M. Levey, Westborough, MA (US); Jennifer Dziczek, Boylston, MA (US); Frank V. Patterson, Exeter, NH (US)

Correspondence Address: WILLIAM C. CAHILL, 155 PARK ONE, 2141 E. HIGHLAND AVENUE, PHOENIX, AZ, 85016, US

(21) Appl. No.: 12/259,428

(22) Filed: Oct. 28, 2008

Related U.S. Application Data
(63) Continuation-in-part of application No. 10/583,292, filed on Mar. 27, 2007.

(51) Int. Cl. A61B 1/01; A61M 25/00

(52) U.S. Cl. 600/114; 604/265

(57) ABSTRACT

A non-invasive device for lubricating and facilitating the insertion and removal of an endoscope into a body cavity. In one embodiment, the device consists of an annular outer tube having an inner diameter sized to permit sliding of an endoscope there through. The outer surface of the outer tube may be suitably textured for improved gripping. A reservoir is positioned on the outer tube and may be compressed to discharge lubricating fluid on a passing endoscope. The tube surface is textured to enhance control and ergonomics and is flexible to allow manipulation, removal and placement. A flange may be positioned at the proximal end of the tube to provide a barrier to movement of excess lubricant onto the central portion of the tube. The invention also comprises the use of a reservoir for lubricants to be applied to the endoscope as it passes through the device and the enhanced control by way of a threaded internal pattern that engages an endoscope outer surface.

### RELATED APPLICATIONS

This application is a continuation-in-part application of U.S. patent application Ser. No. 10/583,292, filed Mar. 27, 2007 (371 filing date), now published application 2008/0051630, and claims priority under 35 U.S.C. § 119(e) to U.S. Provisional Application Ser. No. 60/599,085, entitled “ENDOSCOPIC LUBRICATING AND GRIPPING DEVICE,” filed on Aug. 4, 2004, and to U.S. Provisional Application Ser. No. 60/529,558, entitled “ENDOSCOPIC LUBRICATING DEVICE,” filed on Dec. 16, 2003, which are herein incorporated by reference in their entirety.

### BACKGROUND OF THE INVENTION

1. Field of the Invention

The present invention relates generally to a non-invasive device for facilitating the insertion and removal and lubrication of an endoscope into a body cavity.

2. Description of the Related Art

Endoscopes are used for examining body cavities. By means of a catheter, endoscopes are generally inserted through a body orifice. To facilitate the insertion while minimizing friction, patient pain, and recovery time, a physician will coat the catheter surface with a suitable lubricant, such as a petroleum-based fluid. Primarily, lubricants are applied manually to the endoscope before or during insertion. This manual application has drawbacks, since lubricating the catheter by hand, while simultaneously controlling is time consuming, messy and not always effective. Additionally, lubricating by hand frequently results in an unsatisfactory coating as improper amounts of lubricant may be deposited on the catheter.

Heretofore, attempts have been made to apply a lubricant to the endoscope tube or catheter using appropriate attachments. Typical of these are attachments shown in the Fukuda et. al, U.S. Pat. No. 3,871,358, and the Okada, U.S. Pat. No. 3,805,770. Both of these devices are tubular shaped guides which are inserted at least partially into the large intestines through the sphincter. Since these devices must have larger diameters than the endoscope, they apply more pressure and are likely to cause more distress to the patient than would be the case in which only the endoscope is inserted. U.S. Patent Application 2005/0277809 to Takano discloses an inflexible insertion aide that is partially inserted into a body cavity with a hydrophilic interior surface to assist in lubrication. These devices are inferior because the Food and Drug Administration requires additional procedures to approve any device that is inserted into a patient.

Other devices known in the art that may not require entry into the human body are inferior in that they may require multiple hand operation to ensure proper insertion and withdrawal of a catheter, or other medical guide wire.

For example, some prior art teaches wetting devices used to apply lubrication to a catheter by means of a sponge encircled by a cylindrical support jacket. A wetting substance may be applied when a physician grasps the support jacket between a thumb and forefinger, to supply even pressure to the circumference of the support jacket and thus force lubricant onto the mounted guide wire. The physicians remaining fingers, on a first hand, curl about the distal end of the catheter. In order to properly operate the common devices in the prior art, the physician must use his second hand to stabilize the guide wire in front of the wetting device adjacent to the skin entry point.

Other prior art references teach a shield for use in insertion of a catheter that covers the site and area of the body immediately surrounding the insertion point. The shield is particularly contoured and adapted to maintain itself stably in place upon the areas adjacent to the meatus. These inventions are adapted to be pressed up against the patient's body while a catheter is inserted. Some prior art references teach a separate dispensing container as a reservoir, such as a squeeze bottle, to facilitate the application of a fluid or gas at the insertion point. The prior art does not teach a gripping device for use to manipulate, handle and facilitate insertion or withdrawal of the catheter. In fact, the prior art teaches away from use of a threaded surface on the gripping device. Rather, the prior art suggests a surface must be present on the inserted article in the form of a spiral external grooving, such that the indentations conduct substance from a smooth dispensing appliance through the insertion site and into the body.

The prior art also teaches a sigmoid splint device for endoscopy. This splint device includes an tight distal end to secure and position a catheter as it enters a body cavity. The distal end of the scope is tapered to facilitate insertion of the splint device into the body cavity itself, rendering it inferior as the devices above. Such a device includes an injection port to connect to a syringe or tube as part of its lubrication system. The prior art does not contemplate attachment of the lubrication reservoir. The interior of such a device may include a surface that contains longitudinally extending grooves. These grooves serve to allow flow of fluids from within the splint into the body cavity. The grooves taught by the prior art in no way serve to enhance handling of the splint device, rather are part of the lubrication system.

None of the prior art references disclose a flexible medical-wire grip that allows full control of the wire and lubrication with a single hand, while not requiring insertion of the device into the patient orifice, or contact with the patient body. Furthermore, the prior references do not allow a medical practitioner to use the second, “free”, hand for alternative purposes.

### OBJECTS OF THE INVENTION

It is therefore an object of the present invention to provide a low-cost endoscopic lubricating and gripping device.

It is another object of the present invention to provide a flexible, one-handed gripping and lubricating device for medical wires.

It is a further object of the present invention to provide a gripping device that supports single hand operation to facilitate insertion and withdrawal of an endoscope tube.

It is yet another object of the present invention to provide a endoscopic gripping device that ensures friction handling of a medical wire that is not severely lost when lubrication is present on the medical wire.

It is a still further object of the present invention to provide an endoscopic control handle with an incorporated lube-on-demand feature.

These and other objects of the present invention will become apparent to those skilled in the art as the description proceeds.

### SUMMARY OF THE INVENTION

The present invention provides a novel device that has the dual function of both lubricating and facilitating the controlled insertion, control, and removal of the endoscope tube through the body cavity of any patient. This invention provides an improved means for coating the endoscope as it is being inserted while contemporaneously avoiding increased distress on a patient by avoiding the insertion of the control itself into the body cavity.

In the present invention, there is provided a non-invasive external control for facilitating the insertion and removal of an endoscope, as well as control throughout the procedure, and optionally providing lubrication of the endoscope as the scope is used with the body and within a body cavity. The control is sized to be used with one or two hands, and designed to allow control of the catheter and lubrication allotment with a single hand. In this arrangement, a catheter is placed within the control. The control tube forms a sheath or handle around the medical guide wire. The catheter has a lubricious outer surface that is reactive with a lubricant. The control is made up of a tube with a hollow center to allow the catheter to run along inside the tube. The inner surface of the tube is sized to permit sliding passage of an endoscope and contains a surface pattern to engage and manipulate the catheter running therethrough. The surface pattern may be one or a set of ribs that run along the surface. The surface pattern may also contain a rib that is perpendicular to the first rib to facilitate control of the catheter.

The control tube may be fitted with a reservoir for containing a lubricating fluid that can be applied to the catheter on demand. The reservoir has an opening at the front, and comes to a tapered close around a volume sufficient to hold lubricating fluid. The reservoir may be attached to the control and even share a wall with the control tube. The top side may be formed to allow action by a finger or thumb, whereby pushing on the top side causes the fluid to be released out of the front opening.

The exterior surface of the tube may include a grip texture to allow better handling by a physician with a bare hand or through a latex glove. It is possible to create the entire device (sans catheter) with a single mold.

The present invention also encompasses a control tube that is mainly used for insertion and removal of a catheter into a body cavity. The control tube is outfitted with a reservoir as described previously for dispensing lubricating fluid onto the catheter. If required, the control tube may be outfitted with the necessary internal texture to facilitate more control of the catheter during the procedure. The reservoir may optionally contain a tube, such as a conventional toothpaste tube, and such reservoir tube may include a cap to prevent the unwanted flow of lubricating fluid. To prevent the insertion of the control tube into the body cavity, the control tube has a flange that is larger than the orifice. The control tube and reservoir may be made of a single mold, and the mold may also include the flange.

The present invention also contemplates various methods to insert, control and remove a catheter from a patient's body. In these methods, the catheter is passed through an externally positioned control tube, whereupon lubricating fluid may be deposited on the lubricious outer surface of the catheter in preparation for insertion into a body cavity. The control tube is prevented from being inserted in the patient's body cavity, and can be bent and manipulated with on hand.

### BRIEF DESCRIPTION OF THE DRAWINGS

The present invention may more readily be understood by reference to the accompanying drawings in which:

FIG. 1 is a perspective view of the distal end of the control tube handle;

FIG. 2 is a cross-sectional view taken along the line 2-2 of FIG. 1;

FIG. 3 is a top view of the surface features of the interior surface of the control tube;

FIG. 4 is an enlarged view of a portion of the surface show in FIG. 3;

FIG. 5 is a partial cross-sectional view taken along the line 5-5 of FIG. 4;

FIG. 6 is a perspective view of the control tube;

FIG. 7 is a side view of the control tube;

FIG. 8 is a side view of the proximate end of the control tube;

FIG. 9 is a enlarged view of portion B of FIG. 7;

FIG. 10 is side elevational view of the reservoir tube and cap;

FIG. 11 is an elevational view of the top of an alternative embodiment device;

FIG. 12 is a cross-sectional view taken along line E-E of FIG. 11;

FIG. 13 is a cross-sectional view taken along line G-G of FIG. 12, including both sides of the alternative embodiment of the device;

FIG. 14 is a list indicating device compatibility with market scopes;

FIG. 15 is a table indicating comparative torque forces of the device.

### DETAILED DESCRIPTION OF THE PREFERRED EMBODIMENTS

The foregoing advantages of the present invention will be more fully understood when considered in conjunction with detailed description of the present invention.

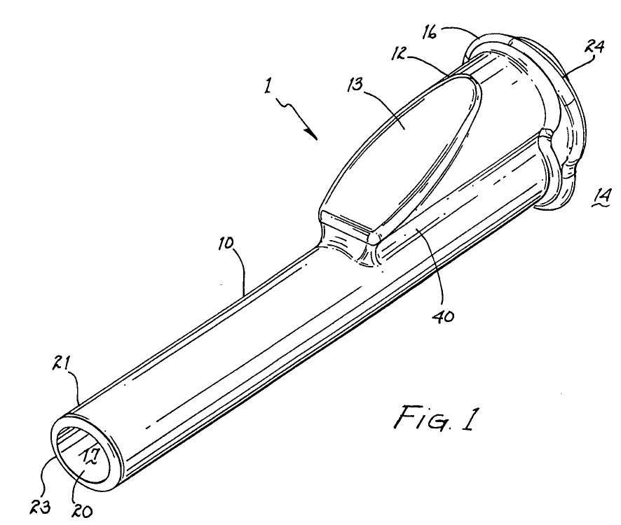

Referring first to FIG. 1, there is illustrated a squeezable cylindrical shell 10 having a length on the order of 3″ to 8″, or sufficiently long enough to permit an operator to grip cylindrical shell 10 in one hand. The shell should be made of a suitable plastic that has sufficient flexibility to permit the operator to grasp the tube, apply pressure onto the endoscope, to control endoscope movement, and cause lubricant to be deposited on the endoscope. The shell should also be soft enough to bend and flex. Endoscope channel 23 allows for the passage of a catheter. The term “catheter” as used herein includes any device that is intended to enter into a human body, cavity, or orifice and includes standard catheters, endoscopes, guide wires and other like devices. When using terms such as endoscope or guide wire, or medical wire, each term is meant to refer to the same group of devices encompassed by the term “catheter” as used herein. Endoscope channel 23 runs through the entire length of control tube 1. Lube channel 24 opens at proximate end 14 and defines a reservoir volume for reservoir 12. Reservoir 12 tapers to a close away from proximal end 14 of control tube 1 such that lubricant will only be discharged near proximal end 14 onto a passing catheter.

Cylindrical shell 10 is fitted with interior diameter 20, and exterior diameter 21, which is greater than interior diameter 20 by the thickness of cylindrical shell 10. Reservoir 12 containing lube channel 24 is paired with cylindrical shell 10 at joint 40. In the preferred embodiment, the entire control tube is cast out of a single mold to ensure structural stability and low cost manufacture.

In a preferred embodiment, suitable polymers for the construction of a single cast control tube 1 include low modulus thermoplastic and thermoset elastomers that are injection moldable and in the range of 30 Shore A durometer to 90 Shore A durometer. Examples of acceptable thermoplastic polymers include: Styrene-ethylene-butylene-styrene (SEBS) copolymers manufactured from GLS Corporation which include Versaflex OM3060-1 and Kraton G27052-1000-00; and styrenic tri-block copolymers of rubber and thermoplastic resin manufactured as the Monprene Brand by Teknor Apex which includes MP-2860E, MP-1304S and MP1643L1. Examples of acceptable thermoset polymers includes polydimethysiloxane silicone manufactured by CRI-SIL Specialty Silicones which includes PRTV-60 (EX-1391). The polymers can be molded with closed or open cell foam structure through the use of appropriate blowing agents. The preferred source for the molded handle is that by CRI-SIL Silicone Technologies LLC of 359 Hill St., Biddeford, Me. 04005. The preferred type of material is CRI-SIL's polydimethylsiloxane with a Platinum Cure at a 10:1 ratio (A to B) and durometer of 60 A. The lot numbers producing the workable device include: P-RTV 60 “A” Lot Mix-9900-01; and P-RTV 60 “B” Lot Mix-9915-01.

In a preferred embodiment, a reservoir 12 extends radially from the cylindrical shell 10 to exit proximal end 14. The reservoir 12 is positioned close to or at the proximal end 14 of the cylindrical shell 10. The reservoir may be integrally molded with the cylindrical shell 10. The reservoir has an outer diameter sized so that it will not ordinarily permit movement of the device into the body cavity. This outer diameter may be formed by flange 16 extending around the circumference of the proximal end. Reservoir 12 is loaded with a suitable lubricant such as a petroleum-based jelly or any other lubricant commonly used for insertion of endoscopes tube through the body cavity.

The preferred surgical lubricant is that made by E. Fougera & Co. of 60 Baylis Rd., Melville, N.Y. 11747. A sterile Bacteriostatic containing Chlorhexidine Gluconate best represents the lubricant for use with control tube 1 and stored in reservoir 12. Those lubricants tested for use with the device are of control numbers: P586 (expiring September 2009); and S270 (expiring February 2010). The sterile surgilube, surgical lubricant, may be used where a sterile water soluble, non-staining lubricating jelly is indicated. The lubricant is non-irritating to the skin, tissues and mucous membranes. The lubricant should not affect surgical instruments, rubber and plastics. “Lubricant” and all like terms refer to fluids, gels, wetting solutions and the like, are to be construed broadly and should not be narrowed to refer only to a jelly or oil product.

The amount of lubricant pre-loaded into the reservoir 12 may vary depending upon the particular uses to which the control is placed. Additional lubricants may be suitably loaded into reservoir 12 with the lubricant distributed.

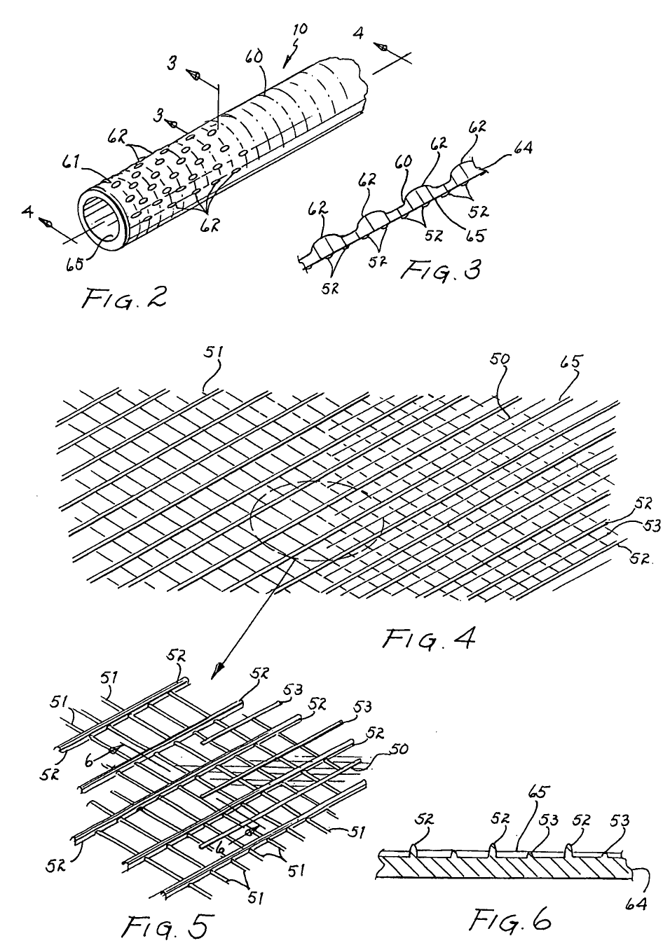

In using the control shown in FIGS. 1-13 to insert an endoscope into a body cavity the operator first threads the catheter through the opening of distal end 17 into control tube 1. Reservoir 12 may be slightly compressed via pressure on thumb panel 13, causing discharge of lubricating fluid as the catheter moves through control tube 1 into intimate contact with the outer surface of the catheter immediately prior to insertion through the body cavity. The operator may use a single finger and or thumb to depress the thumb panel, or may prefer multiple fingers to utilize the thumb panel. When referring to ‘a single finger’, this terminology also refers to a thumb or multiple fingers which may or may not include a thumb, and may or may not be on the same hand. The operator may also flex or thereby squeeze or otherwise force lubricant contained in reservoir 12 onto the outer surface of the catheter prior to its insertion. In conjunction with proximal end 14 and flange 16, reservoir 12 serves a dual purpose of preventing insertion of the control into the body cavity. Reservoir and flange also provide a barrier for preventing excess lubricant from moving backward onto the outer surface of the control tube 1 in contact with the operator's hand.

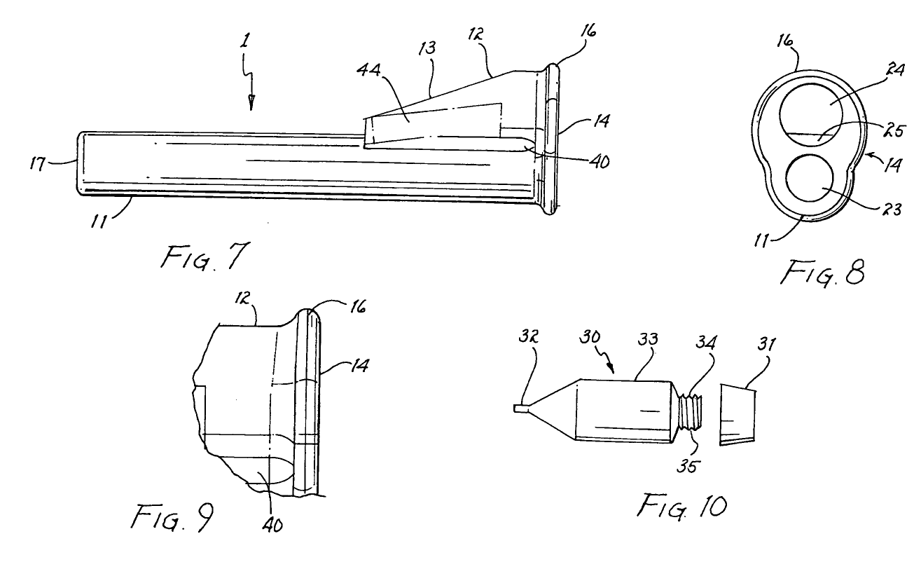

In an alternative embodiment to FIGS. 7-13, the cylindrical shell 10 may be longitudinally cut along a seam 11. The cut may be continuous or a line of perforations such that the control may be removed from an endoscope while the endoscope is inserted into the body cavity. In such a procedure the control is split apart along seam 11 and removed from the endoscope. Since the cylindrical shell 10 is flexible, this step may be completed with ease. Conversely, a fresh control may be applied to the endoscope by spreading the unit along seam 11 and snapping it over the endoscope. Such unit should be made of a resilient plastic to permit the unit to be flexed open to fit around the endoscope tube to retain its shape when so positioned.

Referring to FIG. 2, outer surface 60 of control tube 1 is shown in detail. Outer surface 60 includes grip texture area 61 on the surface where bulbs 62 are deposited. Bulbs 62 function to increase overall surface area of the control tube. Outer surface 60 engages a physician's hand during the medical procedure, and the surface-area-features serve to enhance manual grip and control. The grip texture and bulbs may cover the entire outer surface of the control tube, or may be limited to particular areas to allow multiple grip textures on the surface. Grip texture area 61 extends over a rectangular portion of the tube outer surface; the bulbs of the grip texture area function to increase the surface area of outer surface 60. In the preferred embodiment, grip texture areas encompass two long rectangular sections that extend along the sides of the cylindrical shell.

FIG. 3 demonstrates the relative height of the raised bulbs 62. Outer surface 60 is shown relative to interior surface 65 of control tube shell 64. Shell 64 has a thickness on the order of 1/16″ to a quarter-inch, but will be dependent on the character of the control tube material to maintain proper flexibility and control. The thickness of the shell may vary throughout the length and position of the control tube to accommodate varying components and requirements of flexibility.

Referring to FIGS. 3, 4, 5 and 6, interior surface 65 contains at least one section containing a threaded surface pattern. Internal thread 50 includes a spiraling main rib 52 that runs multiple times around the circumference of interior surface. It is contemplated that the main rib, and other ribs, may encircle the interior surface once, or even less than once, and be of multiple heights as the rib runs along the surface. In the preferred embodiment, the threaded ribs run at an angle between thirty and sixty degrees from vertical and run multiple times around the circumference along the length of cylindrical shell 10. Multiple main ribs are substantially parallel to one another. A first rib should be at forty-five degree angle from the vertical and cross-ribs should be at a negative forty-five degree angle from the vertical such that the ribs are perfectly perpendicular to one another.

In the preferred embodiment, main ribs 52 maintain a nearly constant height along interior surface 65 and circumvent the interior surface multiple times along the entire length of the tube. Ribs extend the entire length of the tube with the brief exception of approximately one-quarter inch on the distal and proximate ends being left flat. Main ribs 52 are evenly spaced apart along the interior surface. One or more support ribs 53 may be positioned between main ribs 52. In the preferred embodiment, the support ribs 53 are symmetrically placed in relation to main ribs 52. A combination of zero, one, and two support ribs are used throughout the length of the control tube as support ribs 53 do not extend the length of the tube. As a first of support ribs 53 is phased out along the tube towards the distal end, the remaining support rib migrates towards the center between two main ribs. Main ribs 52 are extend higher along interior surface 65 than the support ribs. To lower costs, the cross-ribs may extend the length of the control tube. The separation between the main ribs is of the order of one to two millimeters. Generally, the height of the main ribs will be less than their separation along the surface to facilitate surface bending.

Cross-ribs 51, are oriented perpendicular to main ribs 52. The cross-ribs serve to support internal thread 50, and provide ample torque and friction as the physician uses the control tube by twisting to-and-fro on the control tube. When using the term “perpendicular”, the cross-ribs may be placed in such an orientation that they are not exactly ninety-degrees off the main ribs, but at a sufficient angle to permit multiple crossings should the ribs and the length of the control tube be extended indefinitely.

Referring to FIGS. 7 and 9, control tube 1 has reservoir 12 mounted onto cylindrical shell 10. Reservoir 12 is fitted onto cylindrical shell 10 at joint 40. Reservoir 12 is equipped with thumb panel 13 on top and vanity panel 44 on the side. Either panel may encompass a flat plane, or be configured to enhance the effect of the panel or facilitate other functions of the control tube. For instance, thumb panel 13 may contain indentation(s) to facilitate proper placement of a thumb on the reservoir. Additionally, vanity panel 44 may be curved to accommodate a greater volume in the reservoir. Thumb panel or vanity panel may also accommodate further surface textures or serve as an extension of the surface texture of other parts of the device. Flange 16 circumscribes proximal end 14 and serves as the foremost surface of the control tube.

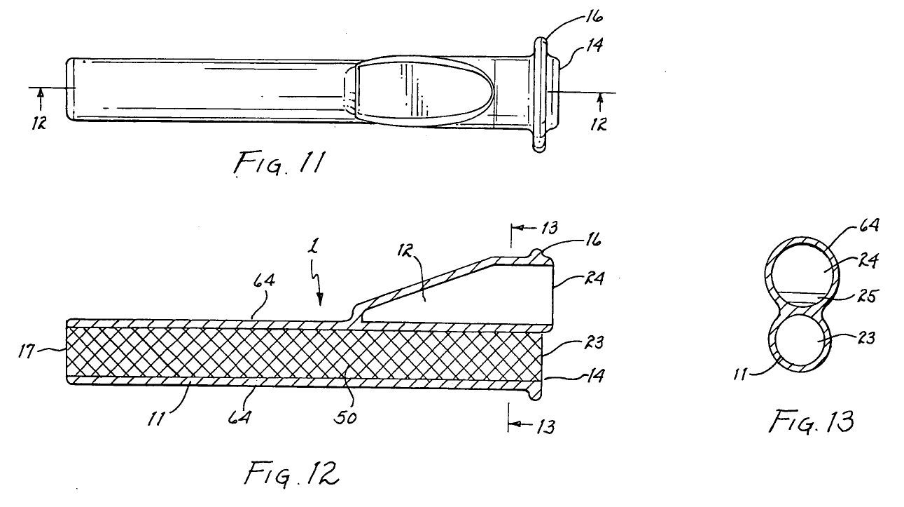

Referring to FIGS. 8 and 13, endoscope channel 23 is situated below lube channel 24. In the preferred embodiment, first channel is sized to accommodate passage of an endoscope with an outside diameter roughly between 8 mm and 12 mm. The control tube may be designed to accommodate other catheters as demand requires. Lube channel 24 allows the flow of lubricating fluid down onto passing endoscope. Pressures exerted on the exterior of the reservoir serve to force fluid out of lube channel 24 and gravity, centripetal force, or surface forces serve to direct the flow of lubricant onto endoscope or patient body. Shelf 25 is included in the reservoir to ensure proper placement of the lubricating fluid container. Flange 16 circumscribes proximal end 14 around both first and second channels.

Referring to FIG. 10, flexible lube tube 30 is shaped as a cinched tube similar to a toothpaste tube. Lube tube 30 is fitted into the reservoir on the control tube. Flexible lube tube 30 contains pre-filled lubricant. When pressure is exerted on the outside of the tube, lubricant is forced through dispensing channel 35. Back end of lube tube is fitted with a crimp seal 32 to fasten the container material together and prevent backflow of lubricant into reservoir. The open end of the lube tube is positioned near the proximal end of the control tube in order to facilitate lubrication flow through the second channel. The crimped end is fitted into the reservoir facing the distal end of the control tube.

Lube tube dispensing channel 35 is fitted with engaging threads 34 on the outside surface for engagement with cap 31 (and complementary internal cap threads, not shown). Cap 31 fits over dispensing channel 35 to prevent flow of lubricating fluid to ease transport, storage, on operation without necessitating immediate lubricating fluid.

Referring to FIGS. 11, 12 and 13, there is shown an alternative embodiment of control tube 1. In this embodiment, flange 16 is positioned somewhat back, so as to no longer form the foremost position on the proximal end 14 of the tube. Flange 16 maintains a forward most position relative to endoscope channel 23. Reservoir 12 and lube channel 24 are molded forward of the flange and jut out beyond endoscope channel 23 to allow better flow of lubricant onto a passing catheter. Internal thread 50 extends the length of endoscope channel 23 from distal end 17 to proximal end 14. Shell 64 is formed around endoscope channel 23 and the entire control tube to secure the outer dimensions of the device.

The proximal end demonstrated in FIG. 13 shows circular opening for both endoscope and lube channels 23 and 24, and varying thickness of shell 64. Shelf 25 may include varying levels or have a slant to better allow a lube tube or other varying or multiple inserts.

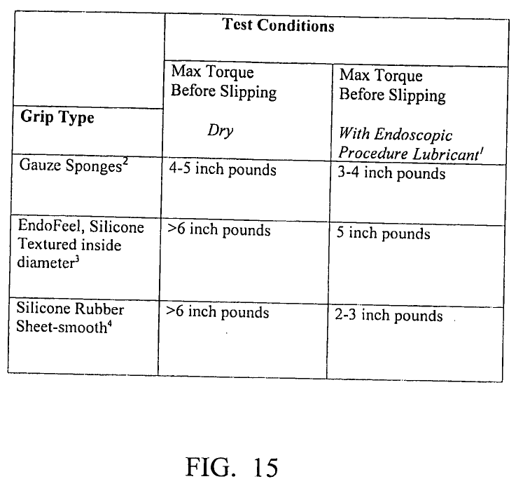

When properly used, the present invention has many advantages over the prior art devices and standard gauze or rubber sheet endoscope grips. In addition to the convenience of the lube-on-demand feature, the control tube has many other advantages. As indicated in FIG. 15, the control tube device entitled ENDOFEEL shows a comparable torque and friction when compared to other devices when dry. However, when lubrication is added to the outer diameter of a passing endoscope, the control tube maintains nearly five inch-pounds of torque on the passing endoscope. This added torque allows substantial pressure to be exerted on the passing catheter given the small size of the control handle device. This substantial torque is accomplished by the selection of materials for the handle, the flexibility of the handle, as well as the interior thread pattern.

In order to use the device, a catheter is prepared for entry into a patient skin entry point, such as an orifice or incision point. The catheter should be sterile to prevent contamination of the subject by way of inserting harmful particles into the patient's body. Given that the control tube is not inserted into the patient's body, it does not necessarily have to be sanitized before the procedure, however, such precautions may be in the best interest of the patient.

The catheter can be threaded through the first channel of the control tube, the proximate end of the catheter first inserted into the distal end of the control tube and passed until it emerges out of the first channel proximate end. Alternatively, the control tube can be mounted at the back end of the catheter, by placing the distal end of the catheter first through the proximate end of the control tube.

Alternatively, should use of the control tube become necessary during a procedure, or should it be more convenient, the control tube may be mounted directly over and onto the catheter. The control tube shaft, or shell, is sliced at the cut line (or seam; see FIG. 12, reference numeral 11) that extends longitudinally down the bottom of the handle. Once sliced, the handle can be “opened” up over the catheter and placed upon the catheter. The handle can then be sealed by a hardening agent, or left open for the remainder of the procedure.

The handle can be held in a single hand, but if preferred, is sized long enough to allow for a second hand on the control tube. A finger (preferably the thumb, but other finger or fingers on the first hand or second hand are envisioned and encompassed in this term) is then used to depress the thumb panel thereby causing outside pressure on the reservoir and thus forcing lubricant out of the second channel. The lubricant can be directed onto the catheter, onto a catheter as it passes, or applied directly to the patient's orifice or exposed body. The control tube should be held by a hand, and neither the handle nor the hand holding the handle should be placed into the patient's orifice. During insertion of the catheter, removal of the catheter, or at any point during the procedure, the control tube may be handled in such a way to exert forward, backward, clockwise rotation, counter-clockwise rotation, or bent along the longitudinal axis with the catheter extending there through to adjust the orientation and position of the catheter.

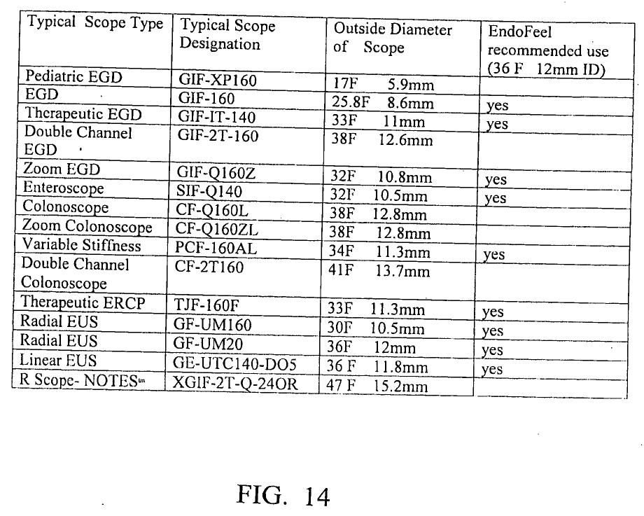

The entire device, excluding any insert such as lube tube 30, can be manufactured from a single mold. This lowers production costs by allowing a single molded piece to be used for application of the control tube. The reservoir volume may be filled directly with lubricant, may be provided with one or more inserts, or left empty to provide a light-weight handling device. The items may be pre-ordered with inserts appropriate for the applicable procedure. FIG. 14 lists device compatibility with typical commercially available endoscopes.

Having thus described at least one illustrative embodiment of the invention, various alterations, modifications, and improvements will readily occur to those skilled in the art. Such alterations, modifications, and improvements are intended to be within and scope of the invention and claims. Accordingly, the foregoing description is by way of example only and is not intended as limiting. The invention is limited only as defined in the following claims and the equivalents thereto.

What is claimed is:

1. A non-invasive flexible single-hand external control for facilitating the insertion, control, and removal of an endoscope into a body cavity comprising:
a) a catheter having a proximate end for insertion into a patient's body and having an opposing distal end, said catheter having an outer surface including a lubricious outer surface that is relatively slippery when activated by a wetting fluid;
b) a longitudinal hollow tube having a central bore extending along the longitudinal axis thereof, said tube having a proximate end and an opposing distal end through which the proximate end of said catheter can be inserted through said distal end, through said bore and emerging from said proximate end of said tube;
c) said tube having an exterior surface along an outside peripheral surface extending partially longitudinally down a shaft of said tube;
d) said tube having an interior surface formed along an inside peripheral surface extending longitudinally through said tube; and
e) said interior surface formed with an internal threaded pattern.

2. The non-invasive flexible external control of claim 1 wherein said internal threaded pattern comprises a spirally threading rib.

3. The non-invasive flexible external control of claim 2 wherein said internal threaded pattern additionally comprises a spirally threading cross-rib running perpendicular from said spirally threaded rib.

4. The non-invasive flexible external control of claim 1 additionally comprising a reservoir positioned distal to said tube bore, said reservoir attached to said tube, said reservoir comprising:
a proximate end, said proximate end having an opening nearer to said tube proximate end,
a distal end, said distal end tapering to a closed end; and
a volume for storage of a lubricating fluid.

5. The non-invasive flexible external control of claim 4 wherein said reservoir additionally comprises:
a bottom side mated with the exterior surface of said tube; and
a top side, opposite said bottom side, having a thumb panel such that exertion of force on said thumb panel increases the pressure within said reservoir causing lubricating fluid to exit said proximate end opening.

6. The non-invasive flexible external control of claim 5 wherein said reservoir additionally comprises a side wall, wherein said sidewall is adapted to display a message.

7. The non-invasive flexible external control of claim 1 wherein said tube exterior surface contains at least one grip texture on the exterior surface for easier manipulation of said tube.

8. The non-invasive flexible external control of claim 1 wherein said tube and said reservoir comprise a single molded frame and said molded frame is of the size to be manipulated by a physician's hand.

9. A non-invasive flexible external control for facilitating the insertion and removal of an endoscope into a body cavity with one hand comprising:
a) a catheter having a proximate end for insertion into a patient's body and having an opposing distal end, said catheter having an outer surface including a lubricious outer surface that is relatively slippery when activated by a wetting fluid;
b) a longitudinal hollow tube having a central bore extending along the longitudinal axis thereof, said tube having a proximate end and an opposing distal end through which the proximate end of said catheter can be inserted through said distal end, through said bore and emerging from said proximate end of said tube, said catheter proximate end positioned for insertion into a patient's body;
c) said tube having an exterior surface formed along a outside circumferential surface extending partially longitudinally down a shaft of said tube;
d) said tube having an interior surface formed along an interior circumferential surface extending longitudinally within the bore of said tube; and
e) a reservoir positioned distal to said tube bore, said reservoir attached to said tube and adapted for manipulation of both said tube and said reservoir with a hand, said reservoir comprising:
a proximate end, said proximate end having an opening nearer to said tube proximate end,
a distal end, said distal end tapering to a closed end, said distal end opposing said proximate end;
a bottom side mated with the exterior surface of said tube;
a top side, opposite said bottom side; and
a containing volume for storage of a lubricating fluid.

10. The non-invasive flexible external control of claim 9 including an internal threaded pattern formed on said interior surface comprising a spirally threading rib.

11. The non-invasive flexible external control of claim 9 including a cinched tube storing lubricating fluid located within said containing volume of said reservoir.

12. The non-invasive flexible external control of claim 11 wherein said reservoir having a thumb panel such that an exertion of force on said thumb panel increases the pressure within said reservoir causing lubricating fluid to exit said cinched tube and out of said proximate end opening.

13. The non-invasive flexible external control of claim 11 wherein said cinched tube having an opening for outflow of fluid, and further including a cap.

14. The non-invasive flexible external control of claim 9 including a flange having a diameter, said flange extending outwardly from said tube wherein the flange diameter is sized to preclude insertion of said tube and said reservoir into the body cavity of a patient.

15. The non-invasive flexible external control of claim 14 wherein said tube, said reservoir, and said flange comprise a single molded structure and said molded structure is of the size to be manipulated by a physician's hand.

16. The non-invasive flexible external control of claim 9 wherein said tube and said reservoir comprise a single molded structure and said molded structure is of the size to be manipulated by a physician's hand.

17. A method for inserting a catheter, the catheter having a lubricious outer surface that is slippery when activated by a wetting fluid, the catheter having a proximate tip to be inserted into a patient's body through a skin entry point, the catheter having a distal tip, by use of a control tube, said method comprising the steps of:
a) inserting one of the proximate end and distal ends of the catheter into at least one of a control tube distal end and a control tube proximate end;
b) forcing the catheter proximate tip through the control tube such that the proximate tip emerges from a control tube proximate end, the control tube proximate end opposing the control tube distal end;
c) holding the control tube with at least one hand;
d) providing a reservoir distal to the control tube;
e) squeezing the reservoir by applying pressure with a finger on the outside of the reservoir in order to excrete lubricating fluid onto the catheter;
f) wetting the catheter lubricious outer surface;
g) inserting the catheter proximate tip into a patient's body; and
h) maintaining the control tube outside of the patient's body.

18. The method as recited in claim 17 including the step of bending the control tube in order to manage at least one of the position and orientation of the catheter.

19. The method as recited in claim 17 wherein said step of holding is accomplished with only one hand and further including the step of operating the control tube with one hand.

20. The method as recited in claim 17 wherein the control tube includes an outer surface, and the control tube includes an inner surface that includes at least one thread, and further including the step of squeezing the control tube by applying pressure on the control tube outer surface, said step of squeezing changing at least the shape or diameter of the control tube so as to engage the control tube inner surface to contact the outer surface of the catheter.

21. A method for controlling the position and orientation of a catheter by use of a control tube, the catheter having a lubricious outer surface that is slippery when activated by a wetting fluid, the catheter having a proximate tip inserted into a patient's body, and wherein the control tube includes an outer surface, and the control tube includes an inner surface, said method comprising the steps of:
a) holding the control tube with at least one hand;
b) providing a reservoir distal to the control tube;
c) squeezing the reservoir by applying pressure with a finger on the outside of the reservoir in order to excrete lubricating fluid onto the catheter;
d) wetting the catheter lubricious outer surface;
e) squeezing the control tube by applying pressure on the control tube outer surface, so as to engage the control tube inner surface to contact the outer surface of the catheter; and
f) maintaining the control tube outside of the patient's body.

22. The method as recited in claim 21 including the step of bending the control tube in order to manage at least one of the position and orientation of the catheter.

23. The method as recited in claim 21 wherein said step of holding is accomplished with only one hand and further including the step of operating the control tube with one hand.

24. The method as recited in claim 21 wherein the control tube inner surface contains at least one thread for engaging the outer surface of the catheter.
